(19) United States
(12) Patent Application Publication
(10) Pub. No.: US 2024/0091491 A1
(43) Pub. Date: Mar. 21, 2024

(54) Inner Support Catheter

(71) Applicant: MicroVention, Inc., Aliso Viejo, CA (US)

(72) Inventors: Nicholas Davis, Mission Viejo, CA (US); Bryan Kan, Orange, CA (US); Joseph Gulachenski, Trabuco Canyon, CA (US)

(73) Assignee: MicroVention, Inc., Aliso Viejo, CA (US)

(21) Appl. No.: 18/525,657

(22) Filed: Nov. 30, 2023

Related U.S. Application Data
(63) Continuation-in-part of application No. PCT/US2023/067044, filed on May 16, 2023.
(60) Provisional application No. 63/342,321, filed on May 16, 2022.
(60) Provisional application No. 63/516,474, filed on Jul. 28, 2023.

(51) Int. Cl. A61M 25/00

(52) U.S. Cl. CPC A61M 25/0041; A61M 25/0045; A61M 2025/0004; A61M 2025/0046

(57) ABSTRACT

The present invention relates to an inner support catheter comprising an elongated catheter body with a distal region having a constrained linear shape and an unconstrained shape. The distal region includes a distal first section and a proximal second section that forms a major curve with the proximal second section in the unconstrained shape. In the unconstrained shape, the proximal second section lies substantially in a first reference plane, while the distal first section is positioned at least partially outside of the first reference plane. This configuration may allow for improved maneuverability and flexibility of the inner support catheter during medical procedures.

### RELATED APPLICATIONS

This application is a continuation-in-part of International Application No. PCT/US2023/067044 filed May 16, 2023 entitled Manufacture and Use of Medical Device Coatings, which claims benefit of and priority to U.S. Provisional Application Ser. No. 63/342,321 filed May 16, 2022 entitled Manufacture and Use of Medical Device Coatings; and this application claims benefit of and priority to U.S. Provisional Application Ser. No. 63/516,474 filed Jul. 28, 2023, entitled Inner Support Catheter, all of which are hereby incorporated herein by reference in their entirety.

### BACKGROUND

The aortic arch is a curved portion of the aorta that connects the ascending and descending aorta. The aortic arch gives rise to three major branches: the brachiocephalic trunk, the left common carotid artery, and the left subclavian artery. These branches supply blood to the head, neck, and upper limbs. The cerebral vasculature, which includes the internal carotid arteries and the vertebral arteries, originates from these branches.

Navigating catheters through the aortic arch to access the cerebral vasculature can be challenging for several reasons. First, the aortic arch has a complex anatomy that varies among individuals. The shape, size, and angle of the aortic arch and its branches can affect the ease and safety of catheterization. Second, the aortic arch is subject to hemodynamic forces that can influence the movement and stability of catheters. The blood flow and pressure in the aortic arch can cause catheters to buckle, kink, or dislodge during navigation. Third, the aortic arch is a potential source of emboli that can cause stroke or other complications. Catheter manipulation in the aortic arch can dislodge plaque or thrombus from the aortic wall or its branches and cause embolic events.

Inner support catheters are devices that can be inserted inside another catheter to provide additional support and stability. Inner support catheters can help navigate catheters through an aortic arch by enhancing their pushability, trackability, and torqueability. Pushability refers to the ability of a catheter to transmit axial force from its proximal end to its distal end. Trackability refers to the ability of a catheter to follow the path of another device or vessel. Torqueability refers to the ability of a catheter to transmit rotational force from its proximal end to its distal end. By improving these properties, inner support catheters can help overcome the anatomical and hemodynamic challenges of catheterization in the aortic arch.

### SUMMARY

In some aspects, the techniques described herein relate to an inner support catheter including: an elongated catheter body including a distal region with a constrained linear shape and an unconstrained shape; wherein the distal region includes a distal first section and a proximal second section that forms a major curve with the proximal second section in the unconstrained shape, wherein the distal first section is distally located relative to the second proximal second section in the constrained linear shape; and, where in the unconstrained shape: the proximal second section lies substantially in a first reference plane; and, the distal first section is positioned at least partially outside of the first reference plane.

In some aspects, the techniques described herein relate to an inner support catheter, wherein a distal tip of the distal first section is positioned proximally of the major curve.

In some aspects, the techniques described herein relate to an inner support catheter, wherein the distal first section is positioned at an angle within an inclusive range of 5 and 45 degrees relative to the first reference plane.

In some aspects, the techniques described herein relate to an inner support catheter, wherein the distal first section is positioned at an angle of about 15 degrees relative to the first reference plane.

In some aspects, the techniques described herein relate to an inner support catheter, wherein a distal tip of the distal first section is positioned at about inclusive range of about 1 about 0.4 cm to 2.0 cm away from a face of the first reference plane.

In some aspects, the techniques described herein relate to an inner support catheter, wherein the distal tip of the distal first section is positioned at a length from the proximal second section that is about parallel to the first reference plane within an inclusive range of about 1.3 cm to about 6.3 cm away from the proximal second section.

In some aspects, the techniques described herein relate to an inner support catheter, wherein the distal first section further includes a first minor curve.

In some aspects, the techniques described herein relate to an inner support catheter, wherein the first minor curve curves in a direction generally away from the proximal second section.

In some aspects, the techniques described herein relate to an inner support catheter, wherein the first minor curve has a curvature less than a curvature of the major curve.

In some aspects, the techniques described herein relate to an inner support catheter, wherein the distal first section is within an inclusive range of about 5 cm to 8 cm.

In some aspects, the techniques described herein relate to an inner support catheter, a low friction coating disposed on the major curve and having a lower friction surface than adjacent areas of the distal region.

In some aspects, the techniques described herein relate to an inner support catheter, a low friction coating disposed on only a portion of the distal region.

In some aspects, the techniques described herein relate to an inner support catheter, including a first friction portion, a second friction portion adjacent the first friction portion, and a third friction portion adjacent the second friction portion; wherein the second friction portion has reduced friction relative to the first friction portion and the third friction portion; and wherein the second friction portion is located over at least the major curve.

In some aspects, the techniques described herein relate to an inner support catheter, wherein the second portion is within an inclusive range of 5-15 times less friction than the first friction portion and the third friction portion.

In some aspects, the techniques described herein relate to an inner support catheter, wherein the inner support catheter has a stiffness at 80 mm from a distal tip of the inner support catheter within a range of about 308.6 gf and 606.5 gf, and a stiffness at 100 mm from the distal tip of the inner support catheter of about 556.4 gm and 1243.8 gf.

In some aspects, the techniques described herein relate to an inner support catheter, wherein the inner support catheter has an average stiffness at 80 mm from a distal tip of the inner support catheter of about 457.6 gf, and an average stiffness at 100 mm from the distal tip of the inner support catheter of about 900.1 gf.

In some aspects, the techniques described herein relate to an inner support catheter, wherein the inner support catheter has a stiffness at about 20 mm within a range of about 7.0 gf and 520.5 gf.

In some aspects, the techniques described herein relate to an inner support catheter, wherein the inner support catheter has an average stiffness at 20 mm of about 192.9 gf.

In some aspects, the techniques described herein relate to an inner support catheter including: an elongated catheter body including a distal region with an unconstrained shape; wherein the distal region includes a first distal section that forms a major curve with a second distal section in the unconstrained shape; and, wherein the unconstrained shape the first distal section is positioned at a non-parallel angle relative to the second distal section and wherein a free distal tip of the distal region is positioned in a generally proximal orientation.

In some aspects, the techniques described herein relate to an inner support catheter including: an elongated catheter body including a distal region with an unconstrained shape means for accessing a carotid artery in an aortic arch; wherein the distal region includes a first distal section that forms a major curve with a second distal section.

In some aspects, the techniques described herein relate to an inner support catheter including: a first portion extending proximally from a distal tip of the catheter; and, a second portion extending proximally from the first portion; wherein the first portion exhibits a higher friction than the second portion; and, wherein the second portion further includes a major curve when the inner support catheter is unconstrained.

In some aspects, the techniques described herein relate to an inner support catheter, further including a third portion extending proximally from the second portion; wherein the third portion exhibits a higher friction than the second portion.

In some aspects, the techniques described herein relate to an inner support catheter, wherein the second portion extends only partially over the major curve, only entirely over the major curve, or proximally and distally beyond the major curve.

In some aspects, the techniques described herein relate to an inner support catheter, wherein the second portion includes a lubricious coating including a hydrophilic polymer on the inner support catheter, and wherein the first portion includes poly(amides), poly(ethylene terephthalate), poly(urethanes), poly(ether sulfones), poly(carbonates), poly(vinyl chloride), copolymers thereof, and derivatives thereof.

In some aspects, the techniques described herein relate to an inner support catheter, wherein the second portion has a length within an inclusive range of about 1 cm to 5 cm.

In some aspects, the techniques described herein relate to an inner support catheter, wherein the first portion exhibits between 5-15 times less friction than the second portion.

In some aspects, the techniques described herein relate to an inner support catheter, wherein the first portion has a frictional value within an inclusive range of about 761 gf to 363 gf, and the second portion 130B has a frictional value of about 29 gf.

In some aspects, the techniques described herein relate to an inner support catheter, wherein the first portion and the third portion have a frictional value within an inclusive range of about 761 gf to 363 gf, and the second portion 130B has a frictional value of about 29 gf.

In some aspects, the techniques described herein relate to an inner support catheter, wherein the third portion is positioned within a reference plane and wherein the first portion is positioned at least partially outside of the first reference plane and wherein a distal tip of the first portion is positioned proximally of the major curve.

In some aspects, the techniques described herein relate to an inner support catheter, including: an elongated catheter body; wherein the elongated catheter body has a stiffness at about 80 mm from a distal tip of the inner support catheter within an inclusive range of about 308.6 gf and 606.5 gf.

In some aspects, the techniques described herein relate to an inner support catheter, wherein the elongated catheter body has a stiffness at about 100 mm from the distal tip within an inclusive range of about 556.4 gm and 1243.8 gf.

In some aspects, the techniques described herein relate to an inner support catheter, wherein the elongated catheter body has a stiffness at about 20 mm within an inclusive range of about 7.0 gf and 520.5 gf.

In some aspects, the techniques described herein relate to an inner support catheter, wherein the elongated catheter body has a stiffness at about 10 mm within an inclusive range of about 12.4 gf and 76.1 gf.

In some aspects, the techniques described herein relate to an inner support catheter, wherein the elongated catheter body has a stiffness at about 5 mm within an inclusive range of about 11.7 gf and 62.9 gf.

In some aspects, the techniques described herein relate to an inner support catheter, wherein the elongated catheter body has an average stiffness at about 80 mm from the distal tip of about 457.6.

In some aspects, the techniques described herein relate to an inner support catheter, wherein the elongated catheter body has an average stiffness of about 100 mm from the distal tip of about 900.1 gf.

In some aspects, the techniques described herein relate to an inner support catheter, wherein the elongated catheter body has an average stiffness of about 20 mm from the distal tip of about 192.9 gf.

In some aspects, the techniques described herein relate to an inner support catheter, wherein the elongated catheter body has an average stiffness of about 10 mm from the distal tip of about 44.2.

In some aspects, the techniques described herein relate to an inner support catheter, wherein the elongated catheter body has an average stiffness of about 5 mm from the distal tip of about 37.3 gf.

In some aspects, the techniques described herein relate to an inner support catheter, further including a major curve and wherein a distal tip of the elongated catheter body is positioned proximally of the major curve.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. Reference is being made to the accompanying drawings, in which:

FIG. 1 illustrates a side view of an example aortic arch of a human.

FIG. 2 illustrates side views of several different examples of aortic arch anatomies of a human.

FIG. 3 illustrates a side view of an inner support catheter in a first rotational position positioned within a plane 100F according to one example of the present disclosure.

FIG. 4 illustrates a side view of the inner support catheter of FIG. 3 in a second rotational position perpendicular to the plane 100F according to one example of the present disclosure.

FIG. 5 illustrates a perspective view of a distal section of the inner support catheter of FIG. 3 according to one example of the present disclosure.

FIG. 6 illustrates a perspective view of a distal section of the inner support catheter of FIG. 3 according to one example of the present disclosure.

FIG. 7 illustrates a perspective view of a distal section of the inner support catheter of FIG. 3 according to one example of the present disclosure.

FIG. 8 illustrates a side view of the inner support catheter of FIG. 3 within an aortic arch according to one example of the present disclosure.

FIG. 9 illustrates a side view of the inner support catheter of FIG. 3 within an aortic arch according to one example of the present disclosure.

FIG. 10 illustrates a side view of the inner support catheter of FIG. 3 according to one example of the present disclosure.

FIG. 11 illustrates a side view of the inner support catheter of FIG. 3 within a larger catheter according to one example of the present disclosure.

FIG. 12 illustrates a graph of a stiffness profile of an inner support catheter according to one example of the present disclosure.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value. Unless otherwise noted, the term “substantially” is defined to mean plus-or-minus 90% of a stated value, state, configuration, or position.

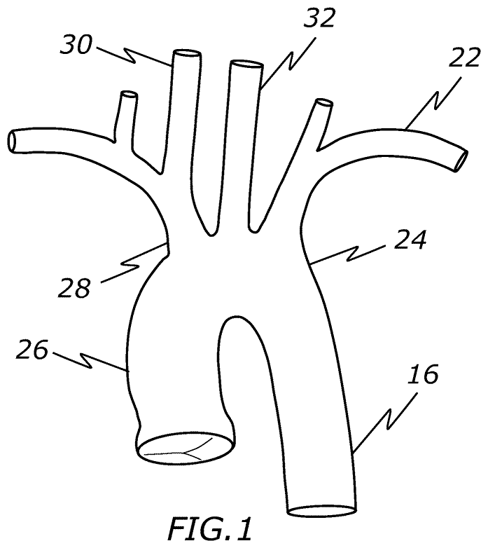

FIG. 1 illustrates a side view of an example aortic arch 24 of a human. Catheter access to upper regions of a patient's body, such as the heart or brain, is most commonly achieved by entering the patient's vasculature at the femoral artery. With reference to FIG. 1, in the example of access to a patient's brain, a catheter (not shown) can be advanced through the descending aorta 16, through the aortic arch 24, and then into either the brachiocephalic artery 28 and the right common carotid artery 30, or into the left common carotid artery 32.

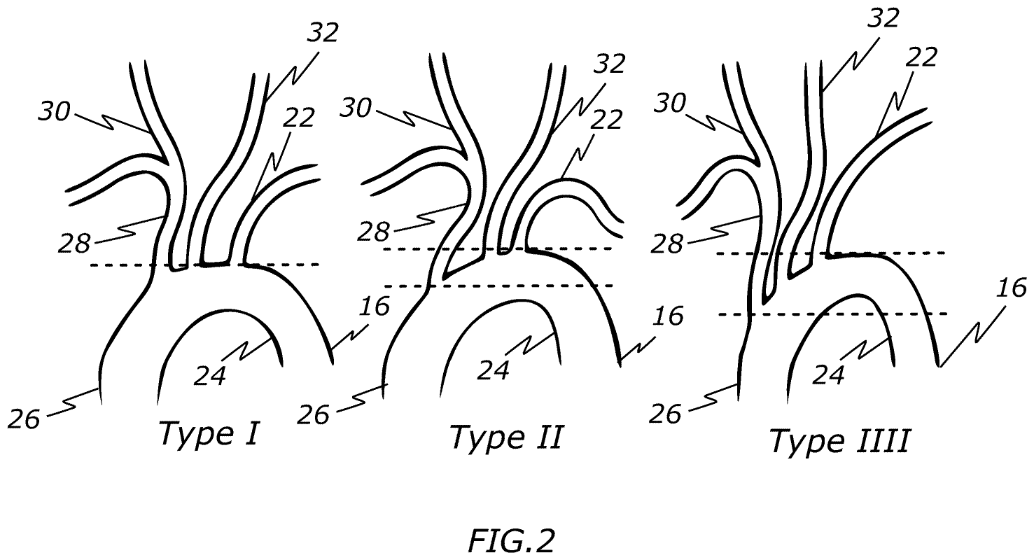

FIG. 2 illustrates side views of several different examples of aortic arch 24 anatomies of a human. Navigating the aortic arch 24 can be further complicated due to the different anatomical variations that position the brachiocephalic artery 28 and left common carotid artery 32 in different locations. For example, FIG. 2 illustrates Type I, Type II, and Type III aortic arch structures. Type I aortic arch anatomies tend to be relatively easier to navigate due to the brachiocephalic artery 28, the left common carotid artery 32, and the left subclavian artery 22 being uniformly located along the aortic arch 24, as noted by the dotted line. However, in Type II and III aortic arch anatomies, the brachiocephalic artery 28 and the left common carotid artery 32 tend to be located lower and closer to the aortic root 26, creating more abrupt angle changes that must be navigated, as noted by the upper and lower dotted lines in each figure.

Inner support catheters (also referred to as access catheters, inner catheters, support catheters, or intermediate catheters) are typically advanced out of a larger catheter and used to direct the larger catheter into a desired vessel from the aortic arch 24. For example, a guide catheter or balloon catheter may be advance into the descending aorta 16 such that its distal end is within the aortic arch 24, and then the inner support catheter is partially advanced out of the distal end of the larger catheter. Since the distal region of the inner catheter has a pre-shaped curve or curves when unconstrained, it may be more easily directed into a desired vessel (e.g., the brachiocephalic artery 28 and the left common carotid artery 32). Once in position, the larger catheter may be advanced over the inner support catheter or a guidewire may be advanced through the inner support catheter and further up the desired vessel (e.g., the right common carotid artery 30 or into the left common carotid artery 32).

The present specification is directed to several features of an inner support catheter that may improve its ability to quickly and easily enter a desired vessel from an aortic arch 24. These features may all be included as part of a single example inner support catheter, or any combination of the features may be included as part of a single inner support catheter (including use of just a single feature). Generally, these features are directed to the three-dimensional unconstrained shape, the location of one or more reduced friction region(s), and a flexibility or stiffness profile of the inner support catheter.

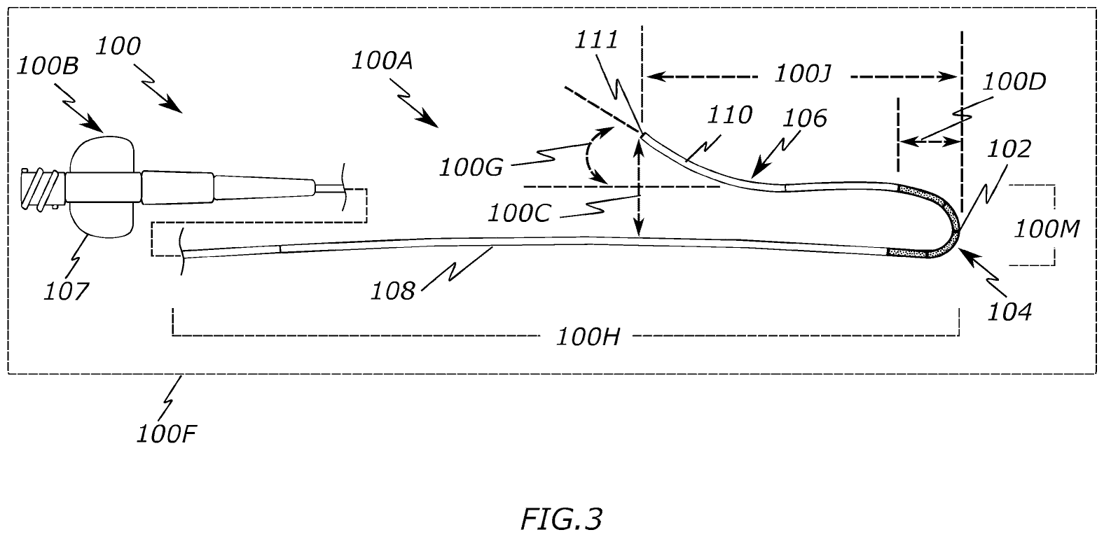

One such feature comprises a distal region of the inner support catheter that curves in at least two different dimensions when unconstrained from an outer catheter (i.e., the curvature does not remain substantially in a single plane). In one example, a distal region of the inner support catheter comprises a first distal section that forms a major curve with a second distal section. The first distal section is distal of a second distal section and forms a major curve with the second distal section when unconstrained. The second distal section lies substantially in a first plane and the first distal section is positioned at least partially outside of the first plane and such that a free distal tip of the distal section is positioned proximally of the major curve. Put another way, the major curve positions the free distal tip in a generally proximal direction and in an orientation that the first distal section is not parallel to the second distal section. In another example, the phrase “proximally of the major curve” may mean that the distal tip is located closer to the proximal end of the catheter and the major curve is located distally with respect to the distal tip. Additional details of the inner support catheter are discussed below with respect to FIGS. 3-12.

Typically, existing inner support catheters only curve within or remain substantially within a single plane which can make accessing some vessels in an aortic arch difficult, particularly for Type II and Type Ill anatomies as previously discussed with regard to FIG. 2. By further including curvature along the distal region of the inner catheter that does not remain within a single plane (e.g., a major curve that positions the free distal tip of the catheter in a generally proximal direction while positioning a distal most region in a non-parallel orientation to proximal adjacent regions of the inner support catheter), different vessels can be more easily accessed, particularly for Type II and Type Ill anatomies. For example, the brachiocephalic artery 28 and the left common carotid artery 32 can be more easily accessed by an inner support catheter comprising a distal region that curves in at least two different dimensions.

Additionally, a region distal of the major curve may further include a minor curve (i.e., a curve with less curvature than the major curve). The minor curve may curve at orientations, angles, and/or planes similar to the major curve (e.g., both major and minor curves may be substantially positioned in the same plane) or may curve at orientations, angles, and/or planes different than that of the major curve (e.g., both major and minor curves may be substantially positioned in the different planes).

Another such feature comprises a low-friction or lubricious coating that is positioned on a portion of a distal region of an inner catheter next to areas of a relatively higher friction of the distal region. While an inner support catheter may particularly benefit from such a low-friction coating or low-friction area, any catheter that includes a pre-shaped or unconstrained curvature that may be deployed through a larger catheter or sheath may also benefit. Hence, this low-friction aspect should not be limited to only inner support catheters.

In one example of an inner support catheter, the coating may be located along only a curved region (e.g., a major curve) and not along the very distal length of the inner catheter. In another example, the coating may be located along only the curved region and some adjacent portions of the curve (e.g., within an inclusive range of about 1 mm to about 10 mm). This may create a first region of relatively increased friction between the distal end of the inner catheter and a short distance thereafter, followed by a second region of relatively decreased friction proximally after the first region and extending over the curved region, followed by a third region of relatively increased friction proximally after the second region. Since inner support catheters tend to have a pre-shaped curve (e.g., a major curve) when unconstrained, when they are constrained or straightened within a larger catheter (e.g., guide catheter or balloon catheter), the curved region tends to provide increased force, and therefore friction, on the inner lumen of the larger catheter. In other words, the curved region tends to press against the walls of the inner lumen of the larger catheter while also forcing regions that are immediately adjacent to the curved region against an opposite side of the wall of the inner lumen of the larger catheter. Hence, the low-friction or lubricious coating may help reduce friction in these areas.

If the low-friction or lubricious coating was continued to the very distal end of the inner support catheter and along much of the proximal portion of the inner support catheter, it may tend to be relatively slippery when located within a vessel off of the aortic arch 24, and therefore when the larger catheter or a guidewire is advanced over/through the inner support catheter, the inner support catheter may tend to slip out of the vessel it is positioned in. Therefore, the higher friction region or regions on one or both sides of the curved region of the inner support catheter may help maintain the inner support catheter in a desired vessel it has been positioned in (e.g., the brachiocephalic artery 28, the right common carotid artery 30, or into the left common carotid artery 32) by providing increased friction against the patient's vessels. Hence, the combination of lower and higher friction regions may help reduce friction when advancing the inner support catheter within a larger catheter and may provide good stability (e.g., remains in place) within a vessel from the aortic arch 24.

In another example, the inner support catheter comprises a stiffness profile that provides certain stiffnesses at certain distances from its distal tip that helps prevent loss of access of the inner support catheter when tracking a guidewire or larger catheter through/over it. For example, some existing inner support catheters include a relatively large jump in stiffness at about 8 cm from their distal tips, which can result in the aforementioned loss of access (i.e., the inner support catheter tends to pull out of a desired vessel it is initially positioned in). By providing a less abrupt transition, the inner support catheter may better maintain access to or position in a desired vessel during a procedure.

As previously discussed, existing inner support catheters only curve within or remain substantially within a single plane which can make accessing some vessels in an aortic arch difficult, particularly for Type II and Type III anatomies as previously discussed with regard to FIG. 2. By including curvature along the distal tip of the inner support catheter so that at least some of a distal region of the inner support catheter does not remain within a single plane (e.g., a major curve that positions the free distal tip of the catheter in a generally proximal direction while positioning a distal most region in a non-parallel orientation to proximal adjacent regions of the inner support catheter), different vessels can be more easily accessed, particularly for Type II and Type III anatomies. For example, the brachiocephalic artery 28 and the left common carotid artery 32 can be more easily accessed.

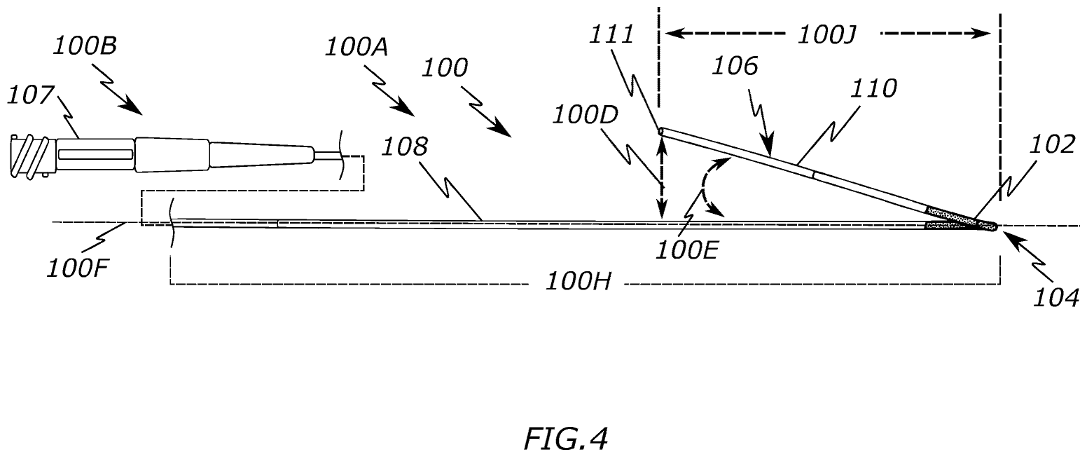

FIGS. 3-7 illustrate an example of an inner support catheter 100 with a curved distal region. FIG. 3 illustrates a side view of the inner support catheter 100 in a first rotational position relative to a plane 100F. FIG. 4 illustrates a side view of the inner support catheter 100 in a second rotational position that is about perpendicular to the position shown in the view of FIG. 3. The reference plane 100F is included in both FIGS. 3 and 4 to help convey the shape of the inner support catheter 100. In FIG. 3, the face of the reference plane 100F faces the viewer and in FIG. 4 the edge of the reference plane 100F faces the viewer.

In one example, the inner support catheter 100 may comprise a generally elongated shape or body that may have a lumen therethrough (e.g., a guidewire lumen) or may not have a lumen. In some examples, the inner support catheter 100 may include a variety of different structural materials that impart different flexibilities, such as inner wire coils, inner braided wires, and outer polymer jackets of different durometer.

Returning to the example of FIG. 3, the inner support catheter 100 may include a distal region 100A and a proximal region 1008. The proximal region 1008 may include a proximal catheter hub 107 or a similar terminal end that allows connection to other medical devices and/or access to the interior lumen, if present. The distal region 100A may extend a distance from the proximal region 1008 and may terminate at a distal tip 111 of the inner support catheter 100. As described further below, the distal region 100A includes an imparted shape that forms when at least part of the distal region 100A is unconstrained (e.g., not constrained by a larger outer catheter). In one example, the distal region may have a length between about 3 cm and about 30 cm when straightened.

In one example, this distal region 100A may include a distal first section 110 and a proximal second section 108. The distal first section 110 and the proximal second section 108 may be unitary with each other where the distal first section 110 is distal and connected to the proximal second section 108 when the inner support catheter 100 is in a constrained linear shape. Adjacent ends of the distal first section 110 and the proximal second section 108 form a major curve with each other. In one example, a major curve may be defined as either the largest curve when the distal region 100A is unconstrained or a curve that bends beyond 45 degrees.

As seen in FIGS. 3 and 4, the first major curve 104 is formed with adjacent ends of the distal first section 110 and the proximal second section 108 and which does not remain substantially in a single plane. In one example, as can be seen in FIG. 3, the first major curve 104 may curve back in a generally proximal direction of about 180 degrees when viewed against the face of the reference plane 100F. However, a variety of different angles may also be possible, such as an inclusive range of 160 degrees to 200 degrees. In more specific examples, the following may be possible: 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 degrees. Hence, the tip 111 of the inner support catheter 100 is positioned generally proximally of the first major curve 104 when the distal region 100A is unconstrained. Put another way, the first major curve 104 repositions the distal tip 111 so that it is not at the distal most location of the inner support catheter 100. In some examples, the tip 111 may face a generally proximal direction as opposed to when the inner support catheter 100 is in a generally linear constrained configuration (e.g., the inner support catheter 100 is forced to take on a straight shape). In one example, an outer diameter 100M at the first major curve 104 is about 0.084 inch.

Referring to FIG. 4, the first major curve 104 may also curve away from the reference plane 100F. FIG. 4 illustrates the reference plane 100F which is rotated at about 90 degrees relative to the view of the reference plane 100F in FIG. 3. In one example, the first major curve 104 forms an angle 100E with the reference plane 100F (e.g., also with the proximal second section 108) within an inclusive range of about 5 degrees to 25 degrees. In some examples, the angle 100E may include the following: about 5, 6, 7, 8, 9, 10, 11, 12, 13, 14, 15, 16, 17, 18, 19, 20, 21, 22, 23, 24, or 25 degrees.

In this respect, the distal first section 110, as well as its distal tip 111, is positioned proximally of the first major curve 104, but is further angled such that the distal first section 110 is non-parallel to the proximal second section 108. In other words, the distal tip 111 of the distal first section 110 is positioned at a larger distance away from the proximal second section 108 than portions of the distal first section 110 at or near the first major curve 104.

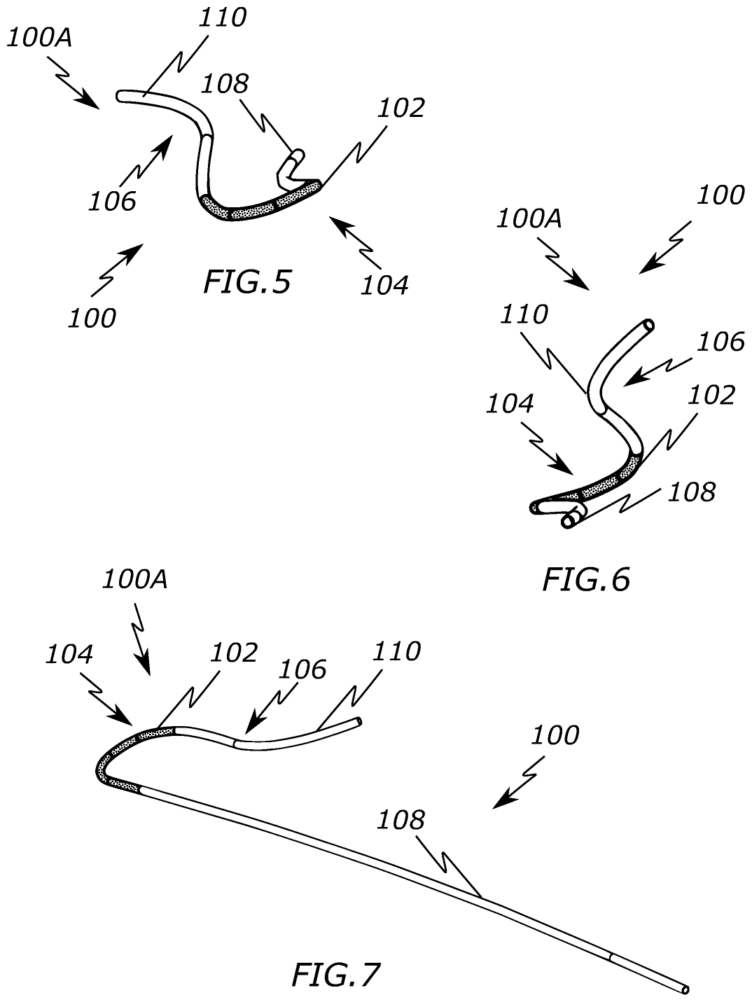

FIGS. 5-7 illustrate other views of the distal region 100A of the inner support catheter 100 that better illustrate the curvature of the first major curve 104. FIG. 5 illustrates a view from a distal vantage point of the distal region 100A when unconstrained such that the first major curve 104 is closest to the viewer and the proximal second section 108 extends away from the viewer. In FIG. 6, the proximal second section 108 generally extends towards the viewer and the first major curve 104 is positioned further away from the viewer. FIG. 7 illustrates another side perspective view of the distal region 100A with the proximal second section 108 being closest to the viewer and the first major curve 104 being furthest from the viewer.

The distal region 100A of the inner support catheter 100 may also include one or more minor curves. In one example, a minor curve is defined as a curve that is less than the first major curve 104 or within an inclusive range of about 1 to 45 degrees relative to the reference plane 100F or a proximal portion of the distal region 100A. In some examples, the angles of the minor curve may also include the following: about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 degrees relative to the reference plane 100F or a proximal portion of the distal region 100A.

Returning to the example of FIGS. 3 and 4, a minor curve 106 may be imparted to a portion of the distal first section 110, creating an angle 100G relative to the reference plane 100F or a proximal portion of the distal first section 110. Angle 100G may be any of the previously disclosed minor curve angles.

In one example, the minor curve 106 may remain substantially within a first plane (plane not shown) such that the entire distal first section 110 may be substantially located in the single plane, which is the case in the example of FIGS. 3 and 4, or the minor curve 106 may further curve out of a first plane (not shown) in which a proximal portion of the distal first section 110 is substantially located, similar to the description of the first major curve 104.

Depending on the length of the distal first section 110 and the curvature of the minor curve 106, the tip 111 of the distal first section 110 may be located at different distances from the proximal second section 108. When viewing from the perspective of FIG. 3, distance 100C (e.g., one component of the distance from between the tip 111 and the proximal second section 108) may be within an inclusive range of about 1.3 cm to about 6.3 cm. More specific examples may include the following: about 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.8, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, or 6.3 cm. When viewing from the perspective of FIG. 4, distance 100D (e.g., another component of the distance from between the tip 111 and the proximal second section 108) may be within an inclusive range of about 0.4 cm to 2.0 cm. More specific examples may include the following: about 0.4, 0.5, 0.6, 0.7, 0.8, 0.9, 1.0, 1.1, 1.2, 1.3, 1.4, 1.5, 1.6, 1.7, 1.8, 1.9, or 2.0 cm.

As further illustrated in FIGS. 3 and 4, in one example, the length 100J between the distal tip 111 of the distal first section 110 and an apex of the curved end of the first major curve 104 may be within an inclusive range of about 5-8 cm. More specific examples may include the following: about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 7.0, 7.1, 7.2, 7.3, 7.4, 7.5, 7.6, 7.7, 7.8, 7.9 or 8.0 cm. A distance 100C between the distal tip 111 and the proximal second section 108 when viewing from position of FIG. 3 may be within an inclusive range of about 2.3-6.3 cm. More specific examples may include the following: about 2.3, 2.4, 2.4, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3 cm.

In one example, the length 100H of the proximal second section 108 may be within an inclusive range of about 0.5 cm to 15 cm. More specific examples may include the following: about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 cm. The proximal second section 108 may be substantially straight or may have a relatively shallow curve along part or all of its length.

In one example, when unconstrained, the inner support catheter may have a length of about 150 cm (+/−3) between a distal tip 111 and the start of a strain relief sleeve connected to a catheter hub 107.

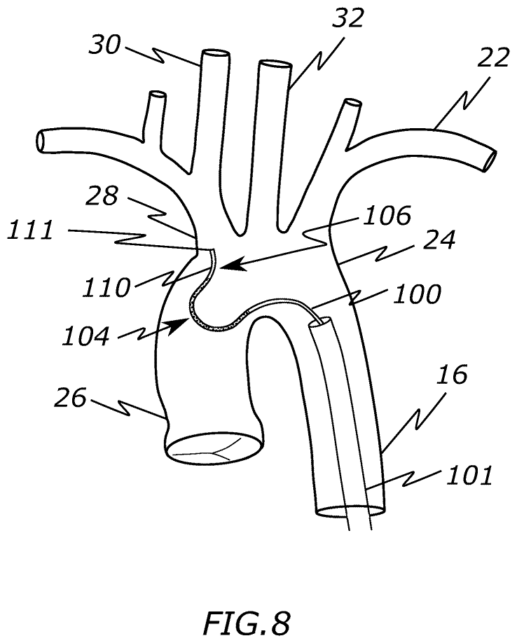

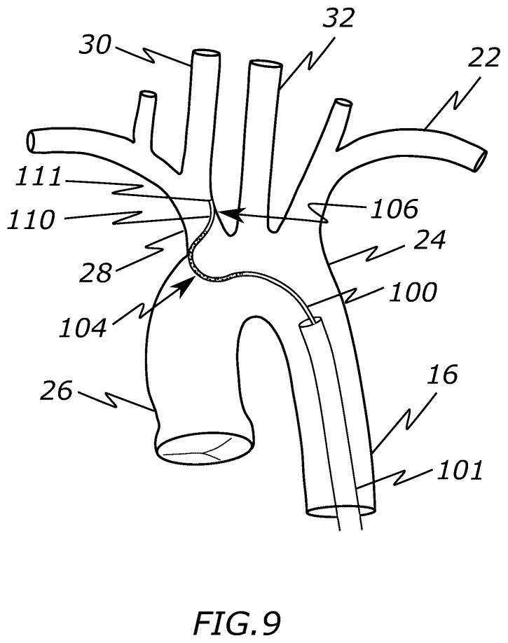

FIGS. 8 and 9 illustrate one example method of use of an example catheter with the previously described unconstrained shape. Referring first to FIG. 8, a larger outer catheter 101 may be advanced near or into an aortic arch 24 of a patient. Next, the inner support catheter 100 may be advanced out of the outer catheter 101. As seen in FIG. 8, the three-dimensional shape of the first major curve 104 helps angle the distal tip 111 towards a desired vessel opening, such as the brachiocephalic artery 28 and the right common carotid artery 30, the left common carotid artery 32, or left subclavian artery 22. The minor curve 106 may also further assist in placement of the distal tip 111. In FIG. 9, the inner support catheter 100 is further advanced into the brachiocephalic artery 28 and toward the right common carotid artery 30. At this point, either the outer catheter 101 may be advanced over the inner support catheter 100 and into the brachiocephalic artery 28 and the right common carotid artery 30, or a guidewire (not shown) may be advanced through the inner support catheter 100 and into the brachiocephalic artery 28 and the right common carotid artery 30. The outer catheter 101 and/or guidewire may be further advanced to a desired location and a treatment may be performed on the patient.

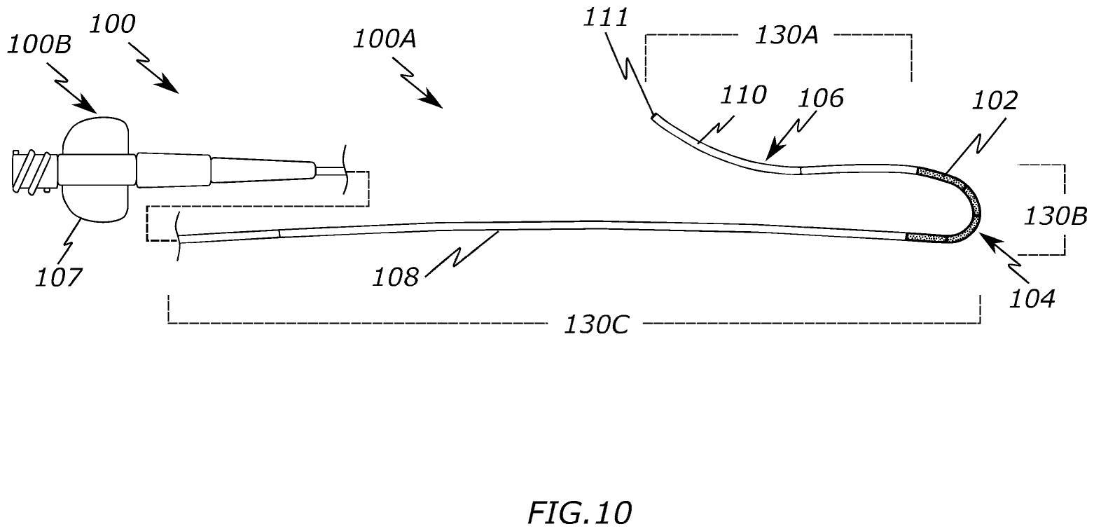

FIG. 10 illustrates a side view of the inner support catheter 100. As previously discussed, in one example, the inner support catheter 100 may include a low-friction or lubricious coating 102 that is positioned on only a portion of a distal region 100A of an inner support catheter 100. In some examples, the areas without the lubricious coating may have a relatively higher coefficient of friction relative to the areas with the lubricious coating. For example, the lubricious coating 102 may be located along only part or all of the first major curve 104 and not along the very distal length of the distal first section 110 or along a proximal length of the proximal second section 108. Alternatively, the lubricious coating 102 may extend a short distance distally and/or proximally beyond the first major curve 104 (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 cm). Optionally, a separate area of the lubricious coating 102 may be included on any additional curves, such as the minor curve 106, in a similar manner described for the major curve 104.

As seen in FIG. 10, these example arrangements of the lubricious coating 102 may include the following example. First, a distal first portion 130A comprises a relatively increased friction between the distal tip 111 of the inner support catheter 100 and a short distance thereafter. That distal first portion 130A is followed by a second portion 130B of lubricious coating 102 of relatively decreased friction proximally after the first portion 130A. The second portion 130B may be positioned over some of, all of, or beyond the first major curve 104. The second portion 130B may be followed by a proximal third portion 130C of relatively increased friction relative to the lubricious coating 102. Further, in one example, a separate section of the lubricious coating 102 may extend along part or all of the minor curve 106 (not shown). In one example, portion may mean a section or region extending along at least some of a length of the inner support catheter 100.

While the example described in FIG. 10 may comprise a lubricious coating 102 to create at least one region with a relatively decreased coefficient of friction, other techniques may also be used to create similar patterns of coefficients of friction. For example, the outer polymer jacket of the inner support catheter 100 may comprise tubular portions that may exhibit different coefficients of friction.

While the example described in FIG. 10 may comprise a lubricious coating 102 that completely encompasses the outer surface or circumference of the inner support catheter 100, other examples may comprise a lubricous coating 102 (or the equivalent jacket material) along only a portion of the outer surface or circumference of the inner support catheter 100. In one example, the lubricous coating 102 (or the equivalent jacket material) may be located along an outer surface of a curved portion and the inner surface of a curved portion may be free of the lubricious coating 102 or may otherwise have a higher coefficient of friction.

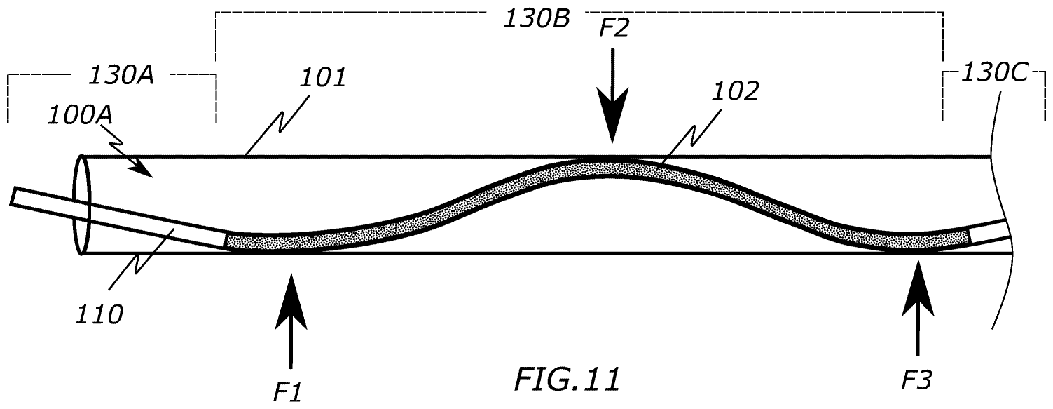

FIG. 11 illustrates a side view of the inner support catheter 100 within a larger catheter 101. In one example, since the inner support catheter 100 may have a pre-shaped curved shape when unconstrained, particularly the first major curve 104 when it is constrained or straightened within a larger catheter 101 (e.g., guide catheter or balloon catheter), portions where the first major curve 104 contact the inner lumen of the larger catheter 101 may create relatively higher levels of friction, and therefore relatively higher levels of force that tends to resist movement, on the inner lumen of the larger catheter 101. The areas with enlarged arrows (e.g., F1, F2, F3) of FIG. 11 illustrate example areas of this relatively higher friction. In some examples, the relatively higher frictional force may be the same or different at the areas F1, F2, and/or F3 and may depend on the degree of curvature of the inner support catheter 100. Hence, the second portion 130B of the lubricious coating 102 may cover at least the area of the middle arrow (e.g., F2) but may also cover the areas of all three arrows, thereby reducing the friction and force needed to advance the inner support catheter 100 through the larger catheter 101.

In some examples, at least the distal first portion 130A of the inner support catheter 100 with relatively higher friction (e.g., non-coated portion) may help maintain the inner support catheter 100 in a desired vessel it has initially been positioned in (e.g., the brachiocephalic artery 28, the right common carotid artery 30, or into the left common carotid artery 32). The proximal third portion 130C may provide further friction with portions of the patient's vessels and therefore further support a position of the inner support catheter 100. Hence, the combination of lower and higher friction sections may help reduce friction when advancing the inner support catheter 100 within a larger catheter 101 and may provide good stability (e.g., remains in place) within a vessel from the aortic arch 24 when the inner support catheter 100 is partially advanced out of the larger catheter 101.

In one example, the distal first portion 130A is within an inclusive range of about 4.8 cm to 6.0 cm or in a specific example of about 5.4 cm. The second portion 130B may have a length within an inclusive range of about 1 cm to 5 cm in a specific example of about 3.0 cm when the inner support catheter 100 is measured straight.

In another example, the distal first portion 130A is within an inclusive range of about 3.8 to 5.0 cm or in a specific example of about 4.4 cm. The second portion 130B may have a length within an inclusive range of about 1 cm to 5 cm in a specific example of about 3.0 cm when the inner support catheter 100 is measured straight.

The distal first portion 130A may exhibit between about 5-15 times less friction than the second portion 130B having the low friction or lubricious coating 102, and in a more specific example about 10 times. For example, the distal first portion 130A may have a frictional value of within an inclusive range of about 761 gf to 363 gf, and the second portion 130B may have a frictional value of about 29 gf. The proximal third portion 130C may exhibit between 5-15 times less friction than the second portion 130B having the low friction or lubricious coating 102, and in a more specific example about 10 times, as well as a frictional value of within an inclusive range of about 761 gf or 363 gf. The frictional values of the distal first portion 130A and proximal third portion 130C may be the same or different. The These frictional gf values may be measured by clamping a specific area and applying a clamping force of about 1 lbs. (+/−0.2 lbs.) of force, pulling the sample through the clamp, and recording the frictional force values.

Generally, the lubricious coating 102 may comprise a variety of different low-friction, lubricious, and/or hydrophilic coatings. One or more such coatings are discussed in PCT/US2023/067044 filed May 16, 2023, which is incorporated by reference in its entirety. The distal first portion 130A and other portions of the inner support catheter 100 may comprise a variety of thermoplastic polymers such as, poly(amides), poly(ethylene terephthalate), poly(urethanes), poly(ether sulfones), poly(carbonates), poly(vinyl chloride), copolymers thereof, and derivatives thereof, such as Peebax.

Disclosed coatings (e.g., lubricious coating 102 or any coatings on remaining portions of the inner support catheter 100) can comprise, for example, multiple coats, such as, for example, a base coat and a top coat. The base coat may function as a “tie” layer between a thermoplastic polymer of the inner support catheter 100 and the top coat. The base coat may be designed to adhere to the catheter and provide binding sites for the attachment of the top coat. The top coat may be designed to adhere to the base coat and provide lubricity to reduce the frictional forces created when the catheter is moved in the vasculature.

The base coat may comprise a polymer that is a copolymer of a first tetrahydrofurfuryl acrylate monomer and at least one other monomer with functional groups capable of further chemical reaction such as hydroxyl, amine, and carboxylic acid groups. The at least one other monomer including hydroxyl groups can be hydroxyethyl methacrylate, hydroxyethyl acrylate, hydroxypropyl acrylate, hydroxypropyl methacrylate, hydroxybutyl acrylate, hydroxybutyl methacrylate, combinations thereof, and derivatives thereof. The at least one other monomer including amine groups can be N-(3-aminopropyl) methacrylamide, 2-aminoethyl methacrylate, 2-aminoethyl methacrylamide, combinations thereof, and derivatives thereof. The at least one other monomer including carboxylic acids can be acrylic acid, methacrylic acid, beta-carboxyethyl acrylate, combinations thereof, and derivatives thereof.

The top coat polymer may comprise a core, hydrophilic polymer that is derivatized with polymerizable groups. The core hydrophilic polymer may be any naturally occurring or synthetic polymer, derivatives thereof and combinations thereof. In some embodiments, the core hydrophilic polymer is at least to some degree, soluble in water.

The structure of the core hydrophilic polymer can be linear or branched, including graft, star, comb, brush, and dendrimer structures.

Polymers used for the top coat may comprise but are not limited to naturally occurring polymers such as proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, and dextran.

Polymers used for the top coat can comprise, but are not limited to synthetic polymers such as poly(ethers), poly(ethylene glycol), poly(ethylene oxide), poly(propylene glycol), poly(lactams), poly(vinylpyrrolidone), poly(acrylates), poly(urethanes), poly(anhydrides), poly(amino acids), poly(carboxylic acids), poly(amides), poly(vinyl alcohol), and poly(phosphazenes).

Molecular weights of the hydrophilic polymers can range from, for example, about 500 amu to about 100,000 amu or from about 1,000 amu to about 40,000 amu.

Reactive groups, such as, but not limited to acrylates and/or methacrylates, can be added to the polymer via any convenient reactive moiety, such as hydroxyls, amines, or carboxylic acids, with a derivatization compound. In some embodiments, the derivatization compound can be a hetero-bifunctional compound. One moiety can react with the hydroxyl, amine, and/or carboxylic acid groups of the copolymer. The other moiety can be an acrylate or methacrylate group.

The derivatization compound may comprise acryloyl chloride, methacryloyl chloride, 2-isocyanatoethyl acrylate, 2-isocyanatoethyl methacrylate, acrylic acid N-hydroxysuccinimide ester, methacrylic acid Nhydroxysuccinimide ester, hetero-bifunctional poly(ethylene glycol) with acrylate and isocyanate groups, combinations thereof, and derivatives thereof.

While the lubricious coating 102 is described as located over portions of the inner support catheter 100 (e.g., the first major curve 104), any catheter and any unconstrained curve, bend, or angle may include such a coating over some of, the entirety of, or even beyond the catheter curve. Note that while curve is used below, it should be considered synonymous with the term bend.

An example curve may have an angle of at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 85 degrees, at least 90 degrees, at least 95 degrees, at least 100 degrees, at least 105 degrees, at least 110 degrees, at least 115 degrees, at least 120 degrees, at least 125 degrees, at least 130 degrees, at least 135 degrees, at least 140 degrees, at least 145 degrees, at least 150 degrees, at least 155 degrees, at least 160 degrees, at least 165 degrees, at least 170 degrees, at least 175 degrees, at least 180 degrees, at least 185 degrees, at least 190 degrees, at least 195 degrees, at least 200 degrees, at least 205 degrees, at least 210 degrees, at least 220 degrees, at least 225 degrees, at least 230 degrees, at least 235 degrees, at least 240 degrees, at least 245 degrees, at least 250 degrees, at least 255 degrees, at least 260 degrees, at least 265 degrees, at least 270 degrees, at least 275 degrees, at least 280 degrees, at least 285 degrees, at least 290 degrees, at least 295 degrees, at least 300 degrees, at least 305 degrees, at least 310 degrees, at least 315 degrees, at least 320 degrees, at least 325 degrees, at least 330 degrees, at least 335 degrees, at least 340 degrees, at least 345 degrees, at least 350 degrees, at least 355 degrees, or the like.

An example curve may have an angle of at most 10 degrees, at most 15 degrees, at most 20 degrees, at most 25 degrees, at most 30 degrees, at most 35 degrees, at most 40 degrees, at most 45 degrees, at most 50 degrees, at most 55 degrees, at most 60 degrees, at most 65 degrees, at most 70 degrees, at most 75 degrees, at most 80 degrees, at most 85 degrees, at most 90 degrees, at most 95 degrees, at most 100 degrees, at most 105 degrees, at most 110 degrees, at most 115 degrees, at most 120 degrees, at most 125 degrees, at most 130 degrees, at most 135 degrees, at most 140 degrees, at most 145 degrees, at most 150 degrees, at most 155 degrees, at most 160 degrees, at most 165 degrees, at most 170 degrees, at most 175 degrees, at most 180 degrees, at most 185 degrees, at most 190 degrees, at most 195 degrees, at most 200 degrees, at most 205 degrees, at most 210 degrees, at most 220 degrees, at most 225 degrees, at most 230 degrees, at most 235 degrees, at most 240 degrees, at most 245 degrees, at most 250 degrees, at most 255 degrees, at most 260 degrees, at most 265 degrees, at most 270 degrees, at most 275 degrees, at most 280 degrees, at most 285 degrees, at most 290 degrees, at most 295 degrees, at most 300 degrees, at most 305 degrees, at most 310 degrees, at most 315 degrees, at most 320 degrees, at most 325 degrees, at most 330 degrees, at most 335 degrees, at most 340 degrees, at most 345 degrees, at most 350 degrees, at most 355 degrees, or the like.

An example curve may have an angle of 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, 185 degrees, 190 degrees, 195 degrees, 200 degrees, 205 degrees, 210 degrees, 220 degrees, 225 degrees, 230 degrees, 235 degrees, 240 degrees, 245 degrees, 250 degrees, 255 degrees, 260 degrees, 265 degrees, 270 degrees, 275 degrees, 280 degrees, 285 degrees, 290 degrees, 295 degrees, 300 degrees, 305 degrees, 310 degrees, 315 degrees, 320 degrees, 325 degrees, 330 degrees, 335 degrees, 340 degrees, 345 degrees, 350 degrees, 355 degrees, or the like.

An example curve may have an angle of between about 10 and 350 degrees, between about 20 and 340 degrees, between about 30 and 330 degrees, between about 40 and 320 degrees, between about 50 and 310 degrees, between about 60 and 300 degrees, between about 70 and 290 degrees, between about 80 and 280 degrees, between about 90 and 270 degrees, between about 100 and 260 degrees, between about 110 and 250 degrees, between about 120 and 240 degrees, between about 130 and 230 degrees, between about 140 and 220 degrees, between about 150 and 210 degrees, between about 160 and 200 degrees, between about 170 and 190 degrees, or the like.

An example curve may have an angle of between about 10 and 40 degrees, between about 20 and 50 degrees, between about 30 and 60 degrees, between about 40 and 70 degrees, between about 50 and 80 degrees, between about 60 and 90 degrees, between about 70 and 100 degrees, between about 80 and 110 degrees, between about 90 and 120 degrees, between about 100 and 130 degrees, between about 110 and 140 degrees, between about 120 and 150 degrees, between about 130 and 160 degrees, between about 140 and 170 degrees, between about 150 and 180 degrees, between about 160 and 190 degrees, between about 170 and 200 degrees, between about 180 and 210 degrees, between about 190 and 220 degrees, between about 200 and 230 degrees, between about 210 and 240 degrees, between about 220 and 250 degrees, between about 230 and 260 degrees, between about 240 and 270 degrees, between about 250 and 280 degrees, between about 260 and 290 degrees, between about 270 and 300 degrees, between about 280 and 310 degrees, between about 290 and 320 degrees, between about 300 and 330 degrees, between about 310 and 340 degrees, between about 320 and 350 degrees, or the like.

As previously discussed, the inner support catheter 100 may have a stiffness profile that provides certain stiffnesses at certain distances from its distal tip that helps prevent loss of access of the inner support catheter when tracking a guidewire through it or larger outer catheter 101 over it. For example, some existing inner support catheters include a relatively large jump in stiffness at about 8 cm from their distal tips, which can result in the aforementioned loss of access. By providing a less abrupt transition, the inner support catheter may better maintain access to a desired vessel during a procedure.

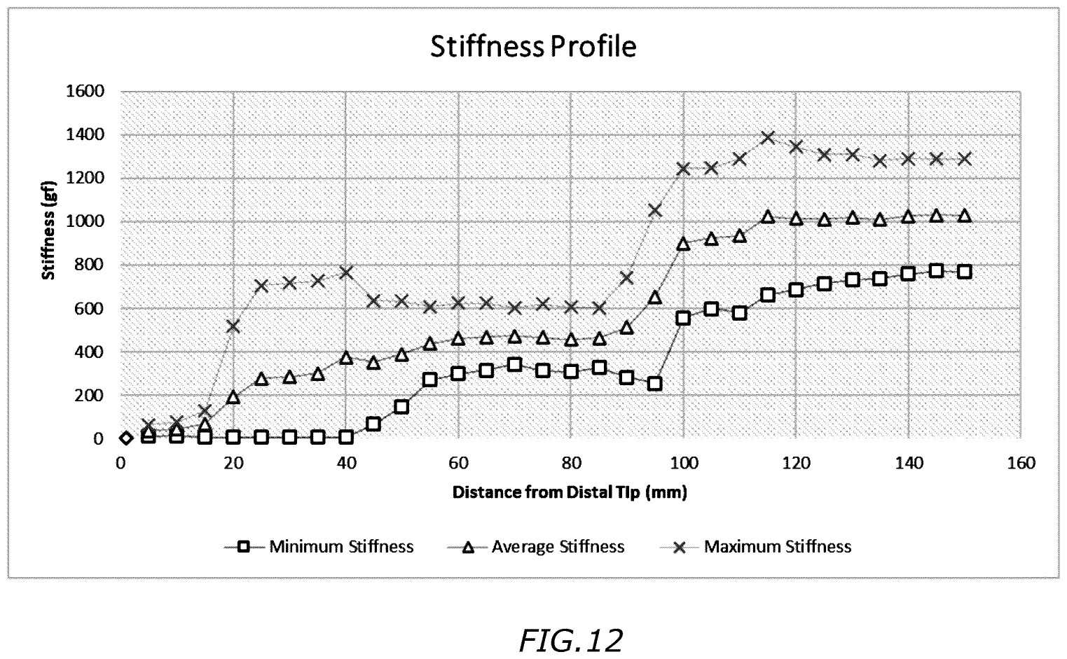

Table 1 below and FIG. 12 illustrate an example stiffness profile for an inner support catheter 100. An average stiffness is provided at different lengths from a distal tip 111 of the inner support catheter 100, but also minimum and maximum stiffnesses are also provided. Hence, these minimum and maximum values may be thought of as a range of stiffness at any specified location in the table or figure.

TABLE 1

| Location | Minimum | Average | Maximum |
|---|---|---|---|
| from distal | Stiffness | Stiffness | Stiffness |
| tip (mm) | (gf) | (gf) | (gf) |
| 5 | 11.7 | 37.3 | 62.9 |
| 10 | 12.4 | 44.2 | 76.1 |
| 15 | 7.0 | 68.5 | 129.9 |
| 20 | 7.0 | 192.9 | 520.5 |
| 25 | 7.0 | 277.2 | 706.1 |
| 30 | 7.0 | 284.9 | 716.8 |
| 35 | 7.0 | 300.5 | 725.9 |
| 40 | 7.0 | 376.5 | 766.3 |
| 45 | 66.7 | 351.3 | 635.8 |
| 50 | 146.7 | 390.1 | 633.5 |
| 55 | 270.6 | 439.0 | 607.3 |
| 60 | 298.6 | 461.6 | 624.7 |
| 65 | 313.9 | 468.8 | 623.6 |
| 70 | 342.1 | 473.2 | 604.2 |
| 75 | 311.8 | 465.9 | 620.0 |
| 80 | 308.6 | 457.6 | 606.5 |
| 85 | 325.9 | 464.6 | 603.3 |
| 90 | 283.6 | 513.5 | 743.3 |
| 95 | 255.8 | 654.1 | 1052.3 |
| 100 | 556.4 | 900.1 | 1243.8 |
| 105 | 598.8 | 923.2 | 1247.7 |
| 110 | 579.1 | 935.3 | 1291.4 |
| 115 | 662.1 | 1024.6 | 1387.2 |
| 120 | 687.2 | 1015.4 | 1343.6 |
| 125 | 715.9 | 1012.9 | 1309.9 |
| 130 | 730.2 | 1020.4 | 1310.6 |
| 135 | 738.5 | 1010.4 | 1282.2 |
| 140 | 759.1 | 1025.2 | 1291.4 |
| 145 | 771.9 | 1030.1 | 1288.4 |
| 150 | 768.1 | 1028.1 | 1288.1 |

In one example, the inner support catheter 100 may have a stiffness at about 100 mm from the distal tip 111 within an inclusive range of about 556.4 gm and 1243.8 gf. In another example, the inner support catheter 100 may have a stiffness at about 80 mm from a distal tip 111 of the inner support catheter within an inclusive range of about 308.6 gf and 606.5 gf. In another example, the inner support catheter 100 may have a stiffness at about 20 mm within an inclusive range of about 7.0 gf and 520.5 gf. In another example, the inner support catheter 100 may have a stiffness at about 10 mm within an inclusive range of about 12.4 gf and 76.1 gf. In another example, the inner support catheter 100 may have a stiffness at about 5 mm within an inclusive range of about 11.7 gf and 62.9 gf.

In another example, the inner support catheter 100 may have an average stiffness at about 100 mm from the distal tip 111 of about 900.1 gf, about 80 mm from the distal tip 111 of about 457.6, at about 20 mm from the distal tip 111 of about 192.9 gf, at about 10 mm from the distal tip 111 of about 44.2, and at about 5 mm from the distal tip 111 of about 37.3 gf.

As previously discussed, the three-dimensional shape, the low friction coating, and the stiffness profile may all be included in the same example catheter, or they may be used in any combination together, including being used alone.

Claim Bank

Clause 1. An inner support catheter comprising: an elongated catheter body comprising a distal region with a constrained linear shape and an unconstrained shape; wherein the distal region comprises a distal first section and a proximal second section that forms a major curve with the proximal second section in the unconstrained shape, wherein the distal first section is distally located relative to the second proximal second section in the constrained linear shape; and, where in the unconstrained shape: the proximal second section lies substantially in a first reference plane; and, the distal first section is positioned at least partially outside of the first reference plane.

Clause 2. The inner support catheter of clause 1, wherein a distal tip of the distal first section is positioned proximally of the major curve.

Clause 3. The inner support catheter of clause 1, wherein the distal first section is positioned at an angle within an inclusive range of 5 and 45 degrees relative to the first reference plane.

Clause 4. The inner support catheter of clause 3, wherein the distal first section is positioned at an angle of about 15 degrees relative to the first reference plane.

Clause 5. The inner support catheter of clause 3, wherein a distal tip of the distal first section is positioned at about inclusive range of about 1 about 0.4 cm to 2.0 cm away from a face of the first reference plane.

Clause 6. The inner support catheter of clause 5, wherein the distal tip of the distal first section is positioned at a length from the proximal second section that is about parallel to the first reference plane within an inclusive range of about 1.3 cm to about 6.3 cm away from the proximal second section.

Clause 7. The inner support catheter of clause 1, wherein the distal first section further comprises a first minor curve.

Clause 8. The inner support catheter of clause 7, wherein the first minor curve curves in a direction generally away from the proximal second section.

Clause 9. The inner support catheter of clause 7, wherein the first minor curve has a curvature less than a curvature of the major curve.

Clause 10. The inner support catheter of clause 1, wherein the distal first section is within an inclusive range of about 5 cm to 8 cm.

Clause 11. The inner support catheter of clause 1, a low friction coating disposed on the major curve and having a lower friction surface than adjacent areas of the distal region.

Clause 12. The inner support catheter of clause 1, a low friction coating disposed on only a portion of the distal region.

Clause 13. The inner support catheter of clause 1, comprising a first friction portion, a second friction portion adjacent the first friction portion, and a third friction portion adjacent the second friction portion; wherein the second friction portion has reduced friction relative to the first friction portion and the third friction portion; and wherein the second friction portion is located over at least the major curve.

Clause 14. The inner support catheter of clause 13, wherein the second portion is within an inclusive range of 5-15 times less friction than the first friction portion and the third friction portion.

Clause 15. The inner support catheter of clause 1, wherein the inner support catheter has a stiffness at 80 mm from a distal tip of the inner support catheter within a range of about 308.6 gf and 606.5 gf, and a stiffness at 100 mm from the distal tip of the inner support catheter of about 556.4 gm and 1243.8 gf.

Clause 16. The inner support catheter of clause 1, wherein the inner support catheter has an average stiffness at 80 mm from a distal tip of the inner support catheter of about 457.6 gf, and an average stiffness at 100 mm from the distal tip of the inner support catheter of about 900.1 gf.

Clause 17. The inner support catheter of clause 15, wherein the inner support catheter has a stiffness at about 20 mm within a range of about 7.0 gf and 520.5 gf.

Clause 18. The inner support catheter of clause 16, wherein the inner support catheter has an average stiffness at 20 mm of about 192.9 gf.

Clause 19. An inner support catheter comprising: an elongated catheter body comprising a distal region with an unconstrained shape; wherein the distal region comprises a first distal section that forms a major curve with a second distal section in the unconstrained shape; and, wherein the unconstrained shape the first distal section is positioned at a non-parallel angle relative to the second distal section and wherein a free distal tip of the distal region is positioned in a generally proximal orientation.

Clause 20. An inner support catheter comprising: an elongated catheter body comprising a distal region with an unconstrained shape means for accessing a carotid artery in an aortic arch; wherein the distal region comprises a first distal section that forms a major curve with a second distal section.

Clause 21. An inner support catheter comprising: a first portion extending proximally from a distal tip of the catheter; and, a second portion extending proximally from the first portion; wherein the first portion exhibits a higher friction than the second portion; and, wherein the second portion further comprises a major curve when the inner support catheter is unconstrained.

Clause 22. The inner support catheter of clause 21, further comprising a third portion extending proximally from the second portion; wherein the third portion exhibits a higher friction than the second portion.

Clause 23. The inner support catheter of clause 22, wherein the second portion extends only partially over the major curve, only entirely over the major curve, or proximally and distally beyond the major curve.

Clause 24. The inner support catheter of clause 21, wherein the second portion comprises a lubricious coating comprising a hydrophilic polymer on the inner support catheter, and wherein the first portion comprises poly(amides), poly(ethylene terephthalate), poly(urethanes), poly(ether sulfones), poly(carbonates), poly(vinyl chloride), copolymers thereof, and derivatives thereof.

Clause 25. The inner support catheter of clause 21, wherein the second portion has a length within an inclusive range of about 1 cm to 5 cm.

Clause 26. The inner support catheter of clause 21, wherein the first portion exhibits between 5-15 times less friction than the second portion.

Clause 27. The inner support catheter of clause 21, wherein the first portion has a frictional value within an inclusive range of about 761 gf to 363 gf, and the second portion 130B has a frictional value of about 29 gf.

Clause 28. The inner support catheter of clause 22, wherein the first portion and the third portion have a frictional value within an inclusive range of about 761 gf to 363 gf, and the second portion 130B has a frictional value of about 29 gf.

Clause 29. The inner support catheter of clause 28, wherein the third portion is positioned within a reference plane and wherein the first portion is positioned at least partially outside of the first reference plane and wherein a distal tip of the first portion is positioned proximally of the major curve.

Clause 30. An inner support catheter, comprising: an elongated catheter body; wherein the elongated catheter body has a stiffness at about 80 mm from a distal tip of the inner support catheter within an inclusive range of about 308.6 gf and 606.5 gf.

Clause 31. The inner support catheter of clause 30, wherein the elongated catheter body has a stiffness at about 100 mm from the distal tip within an inclusive range of about 556.4 gm and 1243.8 gf.

Clause 32. The inner support catheter of clause 31, wherein the elongated catheter body has a stiffness at about 20 mm within an inclusive range of about 7.0 gf and 520.5 gf.

Clause 33. The inner support catheter of clause 32, wherein the elongated catheter body has a stiffness at about 10 mm within an inclusive range of about 12.4 gf and 76.1 gf.

Clause 34. The inner support catheter of clause 33, wherein the elongated catheter body has a stiffness at about 5 mm within an inclusive range of about 11.7 gf and 62.9 gf.

Clause 35. The inner support catheter of clause 30, wherein the elongated catheter body has an average stiffness at about 80 mm from the distal tip of about 457.6.

Clause 36. The inner support catheter of clause 35, wherein the elongated catheter body has an average stiffness of about 100 mm from the distal tip of about 900.1 gf.

Clause 37. The inner support catheter of clause 36, wherein the elongated catheter body has an average stiffness of about 20 mm from the distal tip of about 192.9 gf.

Clause 38. The inner support catheter of clause 37, wherein the elongated catheter body has an average stiffness of about 10 mm from the distal tip of about 44.2.

Clause 39. The inner support catheter of clause 38, wherein the elongated catheter body has an average stiffness of about 5 mm from the distal tip of about 37.3 gf.

Clause 40. The inner support catheter of clause 30, further comprising a major curve and wherein a distal tip of the elongated catheter body is positioned proximally of the major curve.

Clause 41. An inner support catheter having a stiffness profile that includes any of combination of the following values:

| Location | Minimum | Average | Maximum |
|---|---|---|---|
| from distal | Stiffness | Stiffness | Stiffness |
| tip (mm) | (gf) | (gf) | (gf) |
| 5 | 11.7 | 37.3 | 62.9 |
| 10 | 12.4 | 44.2 | 76.1 |
| 15 | 7.0 | 68.5 | 129.9 |
| 20 | 7.0 | 192.9 | 520.5 |
| 25 | 7.0 | 277.2 | 706.1 |
| 30 | 7.0 | 284.9 | 716.8 |
| 35 | 7.0 | 300.5 | 725.9 |
| 40 | 7.0 | 376.5 | 766.3 |
| 45 | 66.7 | 351.3 | 635.8 |
| 50 | 146.7 | 390.1 | 633.5 |
| 55 | 270.6 | 439.0 | 607.3 |
| 60 | 298.6 | 461.6 | 624.7 |
| 65 | 313.9 | 468.8 | 623.6 |
| 70 | 342.1 | 473.2 | 604.2 |
| 75 | 311.8 | 465.9 | 620.0 |
| 80 | 308.6 | 457.6 | 606.5 |
| 85 | 325.9 | 464.6 | 603.3 |
| 90 | 283.6 | 513.5 | 743.3 |
| 95 | 255.8 | 654.1 | 1052.3 |
| 100 | 556.4 | 900.1 | 1243.8 |
| 105 | 598.8 | 923.2 | 1247.7 |
| 110 | 579.1 | 935.3 | 1291.4 |
| 115 | 662.1 | 1024.6 | 1387.2 |
| 120 | 687.2 | 1015.4 | 1343.6 |
| 125 | 715.9 | 1012.9 | 1309.9 |
| 130 | 730.2 | 1020.4 | 1310.6 |
| 135 | 738.5 | 1010.4 | 1282.2 |
| 140 | 759.1 | 1025.2 | 1291.4 |
| 145 | 771.9 | 1030.1 | 1288.4 |
| 150 | 768.1 | 1028.1 | 1288.1 |

What is claimed is:

1. An inner support catheter comprising:
an elongated catheter body comprising a distal region with a constrained linear shape and an unconstrained shape;
wherein the distal region comprises a distal first section and a proximal second section that forms a major curve with the proximal second section in the unconstrained shape, wherein the distal first section is distally located relative to the second proximal second section in the constrained linear shape; and,
where in the unconstrained shape:
the proximal second section lies substantially in a first reference plane; and,
the distal first section is positioned at least partially outside of the first reference plane.

2. The inner support catheter of claim 1, wherein a distal tip of the distal first section is positioned proximally of the major curve.

3. The inner support catheter of claim 1, wherein the distal first section is positioned at an angle within an inclusive range of 5 and 45 degrees relative to the first reference plane.

4. The inner support catheter of claim 3, wherein the distal first section is positioned at an angle of about 15 degrees relative to the first reference plane.

5. The inner support catheter of claim 3, wherein a distal tip of the distal first section is positioned at about inclusive range of about 1 about 0.4 cm to 2.0 cm away from a face of the first reference plane.

6. The inner support catheter of claim 5, wherein the distal tip of the distal first section is positioned at a length from the proximal second section that is about parallel to the first reference plane within an inclusive range of about 1.3 cm to about 6.3 cm away from the proximal second section.

7. The inner support catheter of claim 1, wherein the distal first section further comprises a first minor curve.

8. The inner support catheter of claim 7, wherein the first minor curve curves in a direction generally away from the proximal second section.

9. The inner support catheter of claim 7, wherein the first minor curve has a curvature less than a curvature of the major curve.

10. The inner support catheter of claim 1, wherein the distal first section is within an inclusive range of about 5 cm to 8 cm.

11. The inner support catheter of claim 1, a low friction coating disposed on the major curve and having a lower friction surface than adjacent areas of the distal region.

12. The inner support catheter of claim 1, a low friction coating disposed on only a portion of the distal region.

13. The inner support catheter of claim 1, comprising a first friction portion, a second friction portion adjacent the first friction portion, and a third friction portion adjacent the second friction portion; wherein the second friction portion has reduced friction relative to the first friction portion and the third friction portion; and wherein the second friction portion is located over at least the major curve.

14. The inner support catheter of claim 13, wherein the second portion is within an inclusive range of 5-15 times less friction than the first friction portion and the third friction portion.

15. The inner support catheter of claim 1, wherein the inner support catheter has a stiffness at 80 mm from a distal tip of the inner support catheter within a range of about 308.6 gf and 606.5 gf, and a stiffness at 100 mm from the distal tip of the inner support catheter of about 556.4 gm and 1243.8 gf.

16. The inner support catheter of claim 1, wherein the inner support catheter has an average stiffness at 80 mm from a distal tip of the inner support catheter of about 457.6 gf, and an average stiffness at 100 mm from the distal tip of the inner support catheter of about 900.1 gf.

17. The inner support catheter of claim 15, wherein the inner support catheter has a stiffness at about 20 mm within a range of about 7.0 gf and 520.5 gf.

18. The inner support catheter of claim 16, wherein the inner support catheter has an average stiffness at 20 mm of about 192.9 gf.

19. An inner support catheter comprising:
an elongated catheter body comprising a distal region with an unconstrained shape;
wherein the distal region comprises a first distal section that forms a major curve with a second distal section in the unconstrained shape; and,
wherein the unconstrained shape the first distal section is positioned at a non-parallel angle relative to the second distal section and wherein a free distal tip of the distal region is positioned in a generally proximal orientation.

20. An inner support catheter comprising:
an elongated catheter body comprising a distal region with an unconstrained shape means for accessing a carotid artery in an aortic arch;
wherein the distal region comprises a first distal section that forms a major curve with a second distal section.
